(19) United States
(12) Patent Application Publication
(10) Pub. No.: US 2026/0151643 A1
(43) Pub. Date: Jun. 4, 2026

(54) Flexible Phototherapy Device

(71) Applicant: Haifeng PENG, Jiangsu (CN)

(72) Inventor: Haifeng PENG, Jiangsu (CN)

(21) Appl. No.: 18/966,215

(22) Filed: Dec. 3, 2024

(51) Int. Cl. A61N 5/06

(52) U.S. Cl. CPC A61N 5/06; A61N 2005/0652; A61N 2005/0654; A61N 2005/0659; A61N 2005/0661; A61N 2005/0663

(57) ABSTRACT

A flexible phototherapy device comprises a flexible base and a light-emitting component; the flexible base is provided with at least one installation surface, and the light-emitting component is installed on the installation surface; the light-emitting component comprises a plurality of light-emitting bodies, and a bendable flexible wire is connected between two adjacent light-emitting bodies; when the flexible wire is bent, the relative position between two adjacent light-emitting bodies may be changed; the installation surface is further provided with a positioning element covering the light-emitting component.

### TECHNICAL FIELD

The present disclosure relates to the technical field of phototherapy devices, in particular to a flexible phototherapy device.

### BACKGROUND

Phototherapy is a physical therapy method that uses the radiant energy of light to treat diseases and promote the recovery of the body. Phototherapy began with solar therapy, which was recorded as early as the 2nd century AD. Artificial light source began from the end of 18th century to the middle of 19th century. During this period, visible light, infrared light and ultraviolet light therapy methods were formed one after another, and were widely used and developed in various fields of clinical treatment. For example, ultraviolet rays have anti-inflammatory, analgesic and anti-rickets effects. Infrared rays can improve local blood circulation, promote swelling, relieve pain, reduce muscle tension, relieve muscle spasm. Visible light is the light that human eyes can see, mainly including red light, blue light, blue-violet light and multi-spectral therapy. Red light has anti-aging and fatigue relieving effects. Yellow light, green light and red light have opposite effects. Blue and purple light can be used to treat nuclear jaundice.

With the continuous development of phototherapy, phototherapy devices are constantly changing. The existing U.S. patent application Ser. No. 15/874,973 discloses a “Phototherapy Device”. This patent improves the existing phototherapy device, but there are still some shortcomings. For example, the light-emitting component of this patent is composed of LED lamp panel and LED lamp beads, so that when the phototherapy device is used, the range that the phototherapy device can bend is very small. Therefore, it is not convenient to closely attach the phototherapy device to the contour of the human body, thereby reducing the phototherapy effect.

U.S. Pat. No. 9,895,550 discloses a “Flexible LED light pad for phototherapy”. Although a flexible LED light panel is used to replace the hard LED lamp panel and can be greatly bent. However, because the patent still adopts the form of LED lamp board, and the LED lamp panel has poor bending resistance. After long-term bending, the LED lamp panel is prone to bending fatigue and fracture, which reduces the service life of the phototherapy device. Therefore, the patent cannot solve the above problems.

Therefore, there is a need to put forward a new type of phototherapy device, which can bend greatly, so as to better fit the contour of human body. Moreover, the new phototherapy device is not prone to bending fatigue after long-term bending.

### SUMMARY

The present disclosure provides a flexible phototherapy device to solve the problems raised in the above background technology.

In order to achieve the above object, the present disclosure provides a flexible phototherapy device, which includes a flexible base and a light-emitting component; the flexible base is provided with at least one installation surface, and the light-emitting component is installed on the installation surface; the light-emitting component comprises a plurality of light-emitting bodies, and a bendable flexible wire is connected between two adjacent light-emitting bodies; the relative position between two adjacent light-emitting bodies is variable when the flexible wire is bent; the installation surface is further provided with a positioning element covering the light-emitting component, and the positioning element is configured for keeping the light-emitting bodies evenly distributed on the flexible base; the positioning element is provided with an accommodating area for accommodating the light-emitting bodies, and the light emitted by the light-emitting bodies is able to pass through the positioning element.

The present disclosure further provides a flexible phototherapy device, includes a flexible base and a light-emitting component; the flexible base is provided with at least one installation surface, and the light-emitting component is installed on the installation surface; the light-emitting component comprises a plurality of light-emitting bodies, and a bendable flexible wire is connected between two adjacent light-emitting bodies; the relative position between two adjacent light-emitting bodies is variable when the flexible wire is bent; the installation surface is further provided with a positioning element covering the light-emitting component, and the positioning element is configured for keeping the light-emitting bodies evenly distributed on the flexible base; the positioning element is provided with an accommodating area for accommodating the light-emitting bodies; and the installation surface is further provided with a light-transmitting panel covering the positioning element.

The present disclosure further provides a flexible phototherapy device, which includes a flexible base and a light-emitting component; the flexible base is provided with at least one installation surface, and the light-emitting component is installed on the installation surface; the light-emitting component comprises a plurality of light-emitting bodies, and a bendable flexible wire is connected between two adjacent light-emitting bodies; and the relative position between two adjacent light-emitting bodies is variable when the flexible wire is bent.

### BRIEF DESCRIPTION OF DRAWINGS

FIG. 1 is a schematic structural diagram of the present disclosure.

FIG. 2 is an exploded view of FIG. 1.

FIG. 3 is a partially enlarged schematic view at A of FIG. 2.

FIG. 4 is a sectional view of FIG. 1.

FIG. 5 is a partially enlarged schematic view at B of FIG. 4.

FIG. 6 is a schematic structural view of FIG. 1 with a light-transmitting panel.

FIG. 7 is a sectional view of FIG. 6.

FIG. 8 is a partially enlarged schematic view at C in FIG. 7.

FIG. 9 is an exploded view of FIG. 6.

FIG. 10 is a schematic structural view of the positioning element of the present disclosure when it is a sewing thread.

FIG. 11 is a partially enlarged schematic view at D in FIG. 10.

FIG. 12 is a schematic structural view of a pillow according to the present disclosure.

Reference signs: Flexible base (100); Installation surface (101); Storage bag (102); Sealing structure (1021); Light-emitting component (200); Light-emitting body (201); Flexible wire (202); Controller (203); Connector (204); Positioning element (300); Positioning groove (301); Positioning hole (302); Light-transmitting panel (400).

### DESCRIPTION OF EMBODIMENTS

### Embodiment 1

Please refer to FIG. 1 to FIG. 5.

The present disclosure provides a flexible phototherapy device, which includes a flexible base 100 and a light-emitting component 200. The flexible base 100 has at least one installation surface 101, and the light-emitting component 200 is installed on the installation surface 101. The light-emitting component 200 includes a plurality of light-emitting bodies 201, and a bendable flexible wire 202 is connected between two adjacent light-emitting bodies 201. When the flexible wire 202 is bent, the relative position between two adjacent luminaries 201 may change.

In this embodiment, the flexible base 100 is a cushion and is made of a flexible material, such as sponge, silica gel, latex, cloth and the like. Among them, sponge, as a common flexible material, has excellent shock absorption ability and good air permeability, which can effectively disperse pressure and is not easy to feel tired after long-term use. Silica gel material has the advantages of excellent high temperature resistance, aging resistance, environmental protection and non-toxicity. The flexible base 100 made of silica gel not only has excellent elasticity, and can easily realize various bending or bending actions, but also is easy to clean and maintain, so as to maintain a long-term use experience as new. The flexible base 100 made of latex can not only provide excellent support and air permeability, but also effectively prevent bacteria and dust mites from breeding, which is especially suitable for users who have high requirements on materials. Moreover, the natural elasticity of latex also makes the flexible base 100 more smooth and natural when bending, which is more suitable for human body curves and brings better comfort.

To sum up, with the application of the flexible materials, the flexible base 100 can not only be bent or bent at will to adapt to different use scenarios and individual needs, but also effectively relieve physical stress and improve the overall comfort of users.

In this embodiment, the light-emitting body 201 is an LED lamp bead, and the flexible wire 202 is made of a flexible conductive material, such as copper wire, copper-clad aluminum wire, steel-cored aluminum stranded wire, conductive carbon fiber material and the like. The flexible wire 202 can conduct electricity between two adjacent light-emitting bodies 201. Because the flexible wire 202 also has good toughness and bending resistance, the phototherapy device can bend or bend at will when it is used, and bending fatigue is not easy to occur after bending. Therefore, in this way, the phototherapy device can not only be bent in a large range, but also better fit the contour of human body and improve the phototherapy effect, and can also improve the comfort and use experience of users while ensuring the therapeutic effect. More importantly, these flexible wires 202 can still maintain good electrical conductivity and shape stability after many bends, effectively avoiding the problem of bending fatigue, thus prolonging the service life of the phototherapy device.

The light-emitting body 201 is not limited to LED lamp beads, but in other embodiments (not shown, it may also be incandescent lamp, halogen lamp, fluorescent lamp, plasma lamp and the like. As such, suitable light-emitting bodies 201 can be adopted according to different use scenes and occasions. By selecting different light-emitting bodies 201 in this way, light with different colors and wavelengths can be generated, such as visible light, infrared light, ultraviolet light, etc. For example, ultraviolet rays have anti-inflammatory, analgesic and anti-rickets effects when applied to the human body. Infrared rays can improve local blood circulation, promote swelling, relieve pain, reduce muscle tension, relieve muscle spasm and dry exudation. As such, the phototherapy device can emit specific light for different parts, thus meeting the needs of different users and improving the applicability and product competitiveness of the phototherapy device.

Further, in this embodiment, the flexible wire 202 and the light-emitting bodies 201 are coated with encapsulation glue. By disposing the encapsulation glue, the lead wire and the light-emitting body 201 can be waterproofed, so that the light-emitting component 200 has excellent waterproofness, thereby improving the waterproofness of the phototherapy device. Moreover, the connection between the flexible wire 202 and the light-emitting body 201 is more stable and reliable through the packaging treatment of the packaging adhesive, which not only improves the overall waterproof performance, but also enhances the durability and reliability of the phototherapy device in various complex environments.

The flexible base 100 is not limited to the application form of the cushion in this embodiment, but in other embodiments, it may also be in a form of a hood, so that the phototherapy device can be used in a wearing manner, thereby performing phototherapy on the head. The flexible base 100 may also be designed in a form of a belt, which can be tied around the waist for phototherapy. Of course, in addition to the above application forms, it may be further changed into various wearing forms such as shawls, knee pads, wrist pads, gloves and the like. It may even be integrated into clothing, such as pants, tops, vests, underwear and so on. These methods can allow users to choose the most suitable phototherapy device form according to their own situation according to the different parts and needs of the body, and carry out more accurate and targeted treatment. In addition, for the application of the flexible base 100, the flexible base 100 may also be correspondingly provided with connecting structures, including hook and loop and fasteners. When the phototherapy device is worn on the body, the flexible base 100 can be worn on the body more stably through the connecting structures, without proneness to fall off.

Similarly, the flexible base 100 may also be applied to household products, such as pillows, as shown in FIG. 12. In the application form of the pillow, the light-emitting component 200 is disposed on the surface of the pillow, so that when the user sleeps on the pillow, the user's face and head can be subjected to phototherapy. Of course, it can also take a form of cushion, mattress, quilt, sleeping bag, seat cover, office chair, yoga mat, toilet mat or backrest (not shown). For example, a phototherapy cushion can be designed in an ergonomic style with soft and evenly distributed phototherapy light sources built in. Users can enjoy the benefits of phototherapy simply by putting the cushion on the seat when they are resting, working or watching TV. The material of phototherapy cushion can be soft and breathable fabric to ensure that it will not feel uncomfortable after long-term use. In addition, the phototherapy cushion can also be equipped with an intelligent control system, and users can adjust the intensity and time of phototherapy according to their own needs to realize personalized phototherapy experience. A phototherapy mattress may be designed as a whole mattress or a detachable mattress bedding, and a high-efficiency phototherapy light source is built in to ensure that light can penetrate the mattress evenly and act on the human body. A phototherapy backrest can be designed to conform to the shape of the spine curve, with a built-in phototherapy light source to transmit light to the human body through back contact.

Moreover, the present disclosure is not only used for human phototherapy, but also can be used for animal phototherapy, pet phototherapy or plant phototherapy in other embodiments (not shown), for example, pet phototherapy bandage, pet drying device, plant growth lamp, etc. Moreover, the phototherapy device applied above is also developed and improved based on the flexible base 100 and the light-emitting component 200, so that the phototherapy device can be bent at will, and the phototherapy device can be guaranteed to perform irradiation treatment according to the contours of animals and plants.

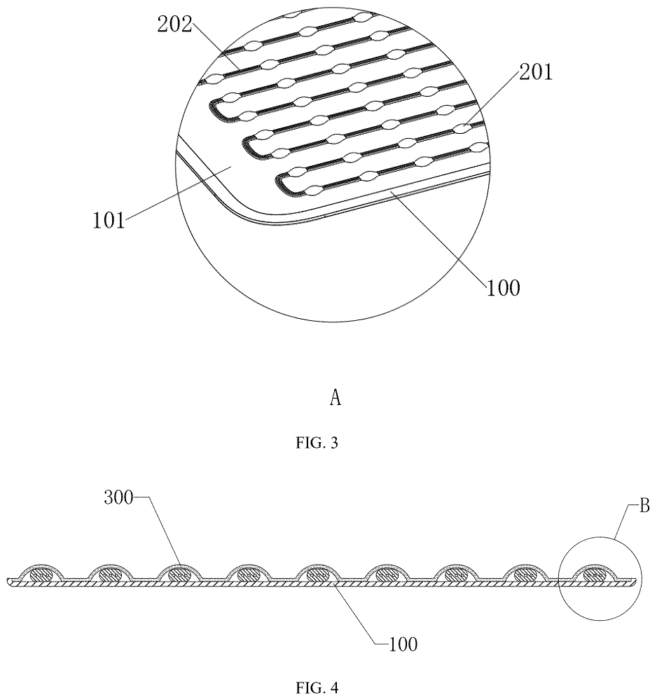

Referring to FIG. 1, FIG. 3 and FIG. 4, the installation surface 101 of the present disclosure is further provided with a positioning element 300 covering the light-emitting component 200, and the positioning element 300 is used to keep the light-emitting bodies 201 evenly distributed on the flexible base 100. The positioning element 300 is provided with an accommodating area for accommodating the light-emitting bodies 201, and the light emitted by the light-emitting body 201 can pass through the positioning element 300.

In this embodiment, the positioning element 300 is made of a flexible light-transmitting material, and the accommodating area is a strip-shaped positioning groove 301, and the light-emitting component 200 is accommodated in the positioning groove 301. In other embodiments (not shown), the positioning element 300 may also be made of a hollow mesh material, such as mesh gauze, so that the light emitted by the light-emitting component 200 can also be emitted through the positioning element 300. As such, different materials can be selected for different application scenarios, and the applicability of phototherapy devices can be improved.

By disposing the positioning groove 301, the light-emitting component 200 can be uniformly disposed on the flexible base 100, which not only improves the overall aesthetics of the phototherapy device, but more importantly, it can ensure that light can be emitted to the parts of the human body that need phototherapy in a more uniform way when using the phototherapy device.

In this embodiment, the positioning groove 301 and the light-emitting component 200 are distributed in an “S” shape. In other embodiments (not shown), the distribution can also be spiral, wave and other shapes. By designing the light-emitting component 200 to be distributed in different shapes, it is ensured that the phototherapy device can be suitable for various flexible bases 100, so that the uniform distribution and in-depth irradiation of light can be realized whether it is a hood, a belt or any other phototherapy device.

In this embodiment, the light-emitting component 200 is fixed on the flexible base 100 or the positioning element 300 by bonding, which makes the light-emitting component 200 simple and convenient to install, reduces the difficulty and cost of processing and manufacturing, and also makes the phototherapy device simple and compact in structure.

In other embodiments, different sizes or different application forms of phototherapy devices can be used, and sewing, clamping, embedding and other fixing methods can also be adopted. For example, the light-emitting component 200 is firmly sewn to the flexible base 100 or a special fabric carrier by sewing, which can not only ensure the stability of connection, but also give consideration to the comfort of wearing. The light-emitting component 200 can be easily clamped or slid into a preset slot on the flexible base 100 by way of clamping, so that the installation can be completed without additional tools, and the convenience of maintenance or replacement is greatly improved. This method is suitable for phototherapy devices that need to adjust the position or intensity of light source frequently. By way of embedding, the sealing performance between the light-emitting component 200 and the flexible base 100 can be improved, which not only ensures the stable installation of the light-emitting component 200, but also effectively prevents the invasion of external factors such as moisture and dust, and improves the protection level and service life of the equipment.

To sum up, the light-emitting component 200 can work stably in different situations by selection of an appropriate securing methods.

In this embodiment, the positioning element 300 is made of colorless and transparent plastic. This ensures that the light emitted by the light-emitting body 201 can pass through the positioning element 300. At the same time, the positioning element 300 can also protect the light-emitting component 200, further improving the waterproof and dustproof capabilities of the phototherapy device. This structure ensures that the light-emitting component 200 inside the device can still work safely even in a humid environment or in the case of accidental water splashing, which greatly prolongs the service life of the phototherapy device. Moreover, due to its excellent waterproof performance, the phototherapy device can be easily washed and cleaned, thus greatly improving the practicability and durability of the phototherapy device.

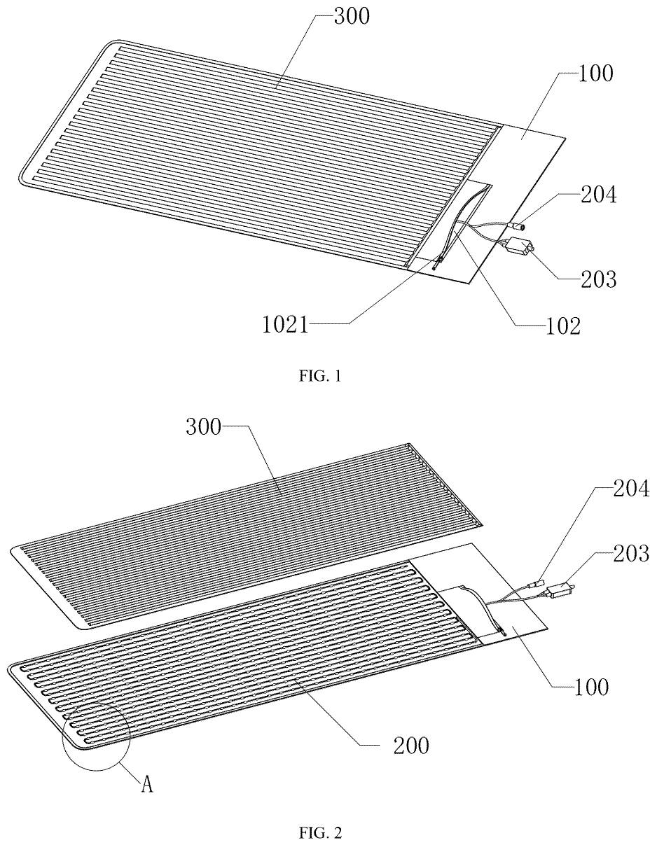

Referring to FIGS. 1 and 2, the light-emitting component 200 of the present disclosure is further provided with a connector 204 and a controller 203, which penetrate through the flexible base 100. The flexible base 100 is provided with a storage bag 102 for accommodating the connector 204 and the controller 203, and the connector 204 and the controller 203 extend out of the storage bag 102.

In this embodiment, the light-emitting component 200 can be connected to an external power supply through the connector 204, thereby providing power for the light-emitting component 200. The controller 203 can control the light-emitting component 200, so as to control the on/off, brightness, light wavelength and the like of the light-emitting component 200.

In this embodiment, by disposing the storage bag 102, when the phototherapy device is not used, the storage bag 102 can store and protect the connector 204 and the controller 203, effectively preventing them from being damaged due to accidental collision or environmental factors. Moreover, the storage bag 102 not only plays a protective role, but also ensures the overall cleanliness and portability of the phototherapy device.

Further, in this embodiment, the storage bag 102 is provided with a sealing structure 1021; When the connector 204 and the controller 203 are stored in the storage bag 102, the sealing structure 1021 is used to seal the storage bag 102.

Preferably, the sealing structure 1021 is a zipper. The mouth of the storage bag 102 can be sealed with the zipper, and the invasion of harmful factors such as external dust and moisture can be prevented. However, the connector 204 and the controller 203 are prevented from falling out of the storage bag 102 and being lost.

Of course, the sealing structure 1021 is not limited to the zipper form in this embodiment, and in other embodiments (not shown), the sealing structure 1021 may also be a magnetic clasp, a hook and loop, a fastener, etc. Selection of different sealing forms facilitates the phototherapy devices to be applied in different scenarios.

### Embodiment 2

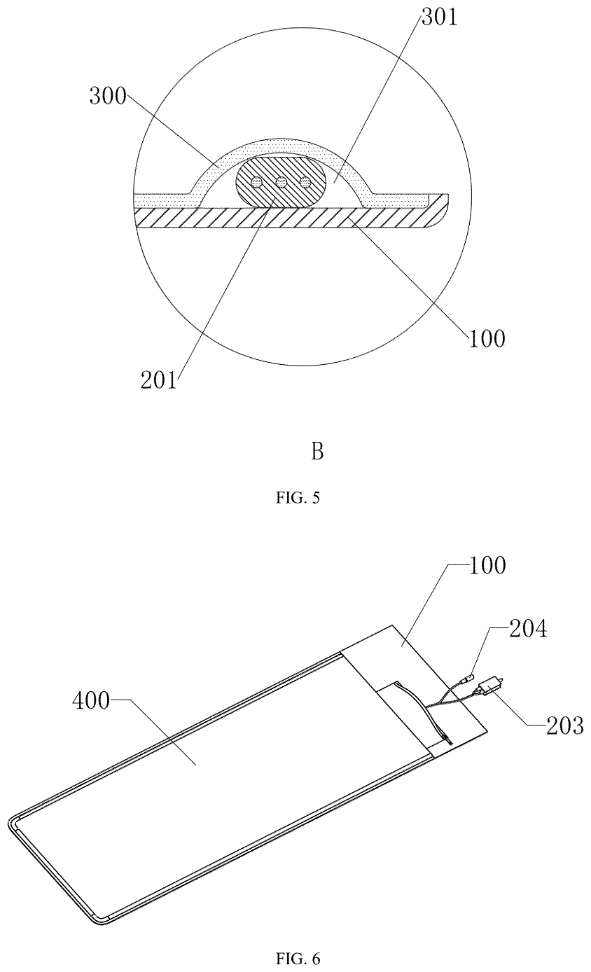

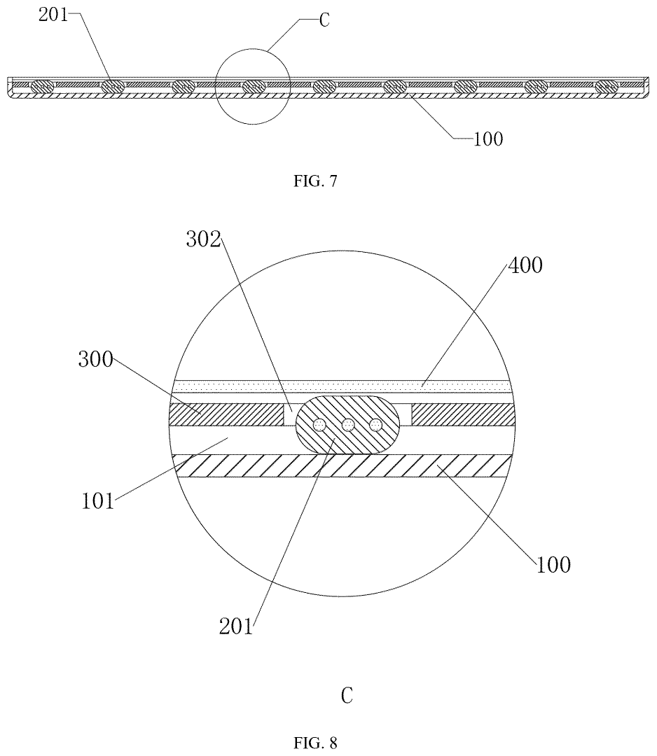

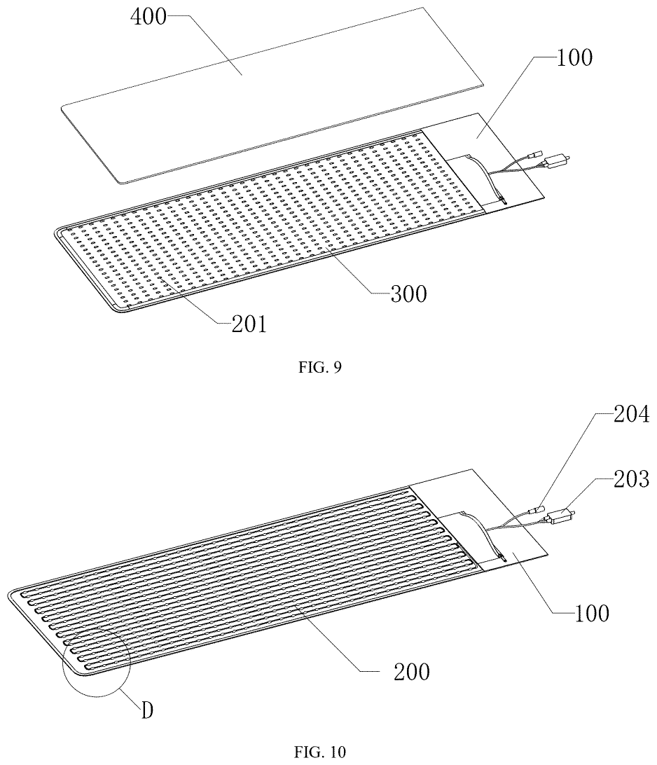

Please refer to FIG. 5 to FIG. 10.

The present disclosure further provides a flexible phototherapy device, which includes a flexible base 100 and a light-emitting component 200. The flexible base 100 has at least one installation surface 101, and the light-emitting component 200 is installed on the installation surface 101. The light-emitting component 200 includes a plurality of light-emitting bodies 201, and a bendable flexible wire 202 is connected between two adjacent light-emitting bodies 201. When the flexible wire 202 is bent, the relative position between two adjacent luminaries 201 may change. The installation surface 101 is further provided with a positioning element 300 covering the light-emitting component 200, and the positioning element 300 is for keeping the light-emitting bodies 201 evenly distributed on the flexible base 100. The positioning element 300 is provided with an accommodating area for accommodating the light-emitting bodies 201.

The above content of this embodiment is basically the same as Embodiment 1, which will not be repeated in this embodiment, except the following:

In this embodiment, the accommodating area is positioning holes 302 uniformly disposed on the positioning element 300. The light-emitting body 201 is embedded in the positioning hole 302, and the flexible wire 202 is located between the positioning element 300 and the flexible base 100. The installation surface 101 is further provided with a light-transmitting panel 400 covering the positioning element 300. The light-transmitting panel 400 is made of a flexible and transparent plastic material.

In this embodiment, the positioning element 300 is made of opaque material, the positioning holes 302 are uniformly formed on the positioning element 300, and with a number corresponding to the number of the light-emitting bodies 201. Therefore, when each light-emitting body 201 is individually embedded in the corresponding positioning hole 302, the light-emitting bodies 201 will be uniformly distributed on the flexible base 100, and at the same time, the light emitted by the light-emitting bodies 201 will be emitted out of the positioning hole 302 and the light-transmitting panel 400, thereby carrying out phototherapy on the irradiated area of the human body.

As such, the positioning element 300 can shield and hide the flexible wire 202, making the whole phototherapy device more beautiful. The light-transmitting panel 400 can protect the light-emitting component 200, and play a waterproof and dustproof role.

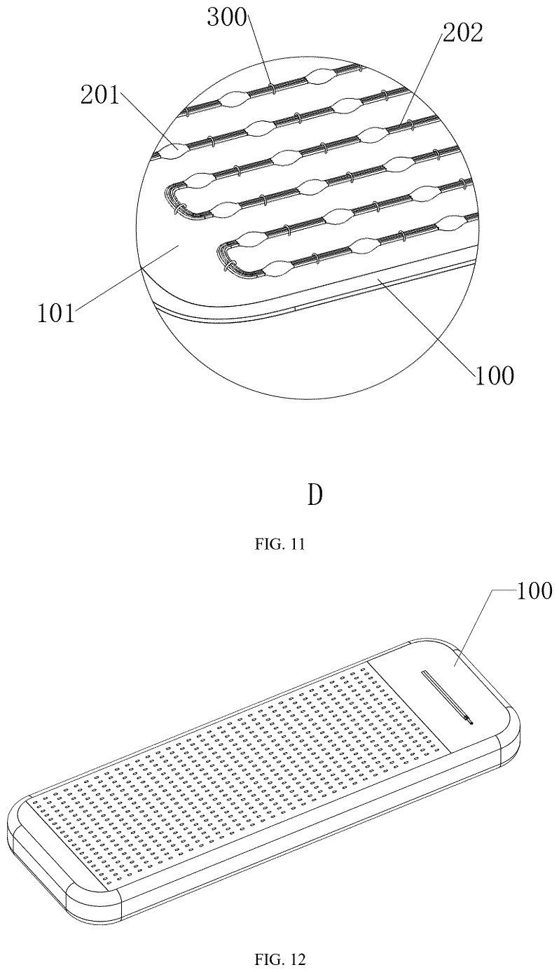

The positioning element 300 is not limited to the above two embodiments, but in other embodiments, as shown in FIGS. 10 and 11. The positioning element 300 is a sewing thread, and the flexible wire 202 is sutured together with the flexible base 100 through the sewing thread, so as to secure the light-emitting component 200 on the flexible base 100. At the same time, the light-emitting bodies 201 are uniformly distributed on the flexible base 100. By way of sewing and positioning, the processing and manufacturing cost of phototherapy device can be further reduced. Moreover, because of this positioning and fixing method, the structure is relatively simple, so it can be applied to phototherapy scenes that do not need direct contact with the human body except contact phototherapy scenes. Of course, the positioning element 300 is not limited to the form of sewing thread, and in other ways, any other form suitable for securing the flexible wire 202 can be used, such as ribbon, adhesive tape, glue bonding and the like.

What is claimed is:

1. A flexible phototherapy device, comprising a flexible base and a light-emitting component, wherein
the flexible base is provided with at least one installation surface, and the light-emitting component is installed on the installation surface; and
the light-emitting component comprises a plurality of light-emitting bodies, and a bendable flexible wire is connected between two adjacent light-emitting bodies; and
the relative position between two adjacent light-emitting bodies is variable when the flexible wire is bent; and
the installation surface is further provided with a positioning element covering the light-emitting component, and the positioning element is configured for keeping the light-emitting bodies evenly distributed on the flexible base; and
the positioning element is provided with an accommodating area for accommodating the light-emitting bodies, and the light emitted by the light-emitting bodies is able to pass through the positioning element.

2. The flexible phototherapy device according to claim 1, wherein the positioning element is made of a flexible light-transmitting material or a hollow mesh material.

3. The flexible phototherapy device according to claim 1, wherein the accommodating area is a strip-shaped positioning groove, and the light-emitting component is accommodated in the positioning groove.

4. The flexible phototherapy device according to claim 1, wherein the light-emitting component is further provided with a connector and a controller which penetrate through the flexible base.

5. The flexible phototherapy device according to claim 4, wherein the flexible base is provided with a storage bag for storing the connector and the controller, and the connector and the controller extend out of the storage bag.

6. The flexible phototherapy device according to claim 5, wherein the storage bag is provided with a sealing structure; and
when the connector and the controller are stored in the storage bag, the sealing structure is configured for sealing the storage bag.

7. The flexible phototherapy device according to claim 6, wherein the sealing structure is a zipper, a hook and loop or a fastener.

8. The flexible phototherapy device according to claim 1, wherein the flexible lead and the light-emitting bodies are coated with encapsulation glue.

9. A flexible phototherapy device, comprising a flexible base and a light-emitting component, wherein
the flexible base is provided with at least one installation surface, and the light-emitting component is installed on the installation surface; and
the light-emitting component comprises a plurality of light-emitting bodies, and a bendable flexible wire is connected between two adjacent light-emitting bodies; and
the relative position between two adjacent light-emitting bodies is variable when the flexible wire is bent; and
the installation surface is further provided with a positioning element covering the light-emitting component, and the positioning element is configured for keeping the light-emitting bodies evenly distributed on the flexible base; and
the positioning element is provided with an accommodating area for accommodating the light-emitting bodies; and
the installation surface is further provided with a light-transmitting panel covering the positioning element.

10. The flexible phototherapy device according to claim 9, wherein the light-transmitting panel is made of a flexible light-transmitting material.

11. The flexible phototherapy device according to claim 9, wherein the accommodating area is a plurality of positioning holes uniformly disposed on the positioning element.

12. The flexible phototherapy device according to claim 11, wherein the light-emitting bodies are embedded in the positioning holes, and the flexible wire is located between the positioning element and the flexible base.

13. A flexible phototherapy device, comprising a flexible base and a light-emitting component, wherein
the flexible base is provided with at least one installation surface, and the light-emitting component is installed on the installation surface; and
the light-emitting component comprises a plurality of light-emitting bodies, and a bendable flexible wire is connected between two adjacent light-emitting bodies; and
the relative position between two adjacent light-emitting bodies is variable when the flexible wire is bent.

14. The flexible phototherapy device according to claim 13, wherein the flexible lead and the light-emitting body are coated with encapsulation glue.

15. The flexible phototherapy device according to claim 13, wherein the installation surface is further provided with a positioning element covering the light-emitting component, and the positioning element is configured for keeping the light-emitting bodies evenly distributed on the flexible base; and
the positioning element is provided with an accommodating area for accommodating the light-emitting bodies.

16. The flexible phototherapy device according to claim 15, wherein both the positioning element and the flexible base are made of flexible materials.

17. The flexible phototherapy device according to claim 15, wherein the accommodating area is a strip-shaped positioning groove, and the light-emitting component is accommodated in the positioning groove.

18. The flexible phototherapy device according to claim 15, wherein the accommodating area is a plurality of positioning holes uniformly disposed on the positioning element.

19. The flexible phototherapy device according to claim 18, wherein the installation surface is further provided with a light-transmitting panel covering the positioning element.

20. The flexible phototherapy device according to claim 19, wherein the light-transmitting panel is made of a flexible and transparent material.
